(19) United States
(12) Patent Application Publication
(10) Pub. No.: US 2017/0082180 A1
(43) Pub. Date: Mar. 23, 2017

(54) Continuously Variable Planetary Transmission

(71) Applicant: Universite Catholique De Louvain, Louvain-la-Neuve (BE)

(72) Inventors: Christophe Everarts, Lasne (BE); Bruno Dehez, Liernu (BE); Renaud Ronsse, Louvain-la-Neuve (BE)

(73) Assignee: Universite Catholique De Louvain, Louvain-La-Neuve (BE)

(21) Appl. No.: 15/308,830

(22) PCT Filed: Apr. 30, 2015
(86) PCT No.: PCT/EP2015/059572

§ 371 Date: Nov. 3, 2016

(30) Foreign Application Priority Data
May 5, 2014 (GB) 1407887.7

(51) Int. Cl. F16H 37/02; A61F 2/42; A61F 2/66; F16H 3/46; F16H 15/50

(52) U.S. Cl. CPC F16H 37/022; F16H 3/46; F16H 15/506; A61F 2/6607; A61F 2/4202; A61F 2002/6836

(57) ABSTRACT

Transmission (10) comprising a sun (1), a planet carrier (4), a first planet (21) having a first axis of revolution (41) and a first lateral surface (31) that is nonparallel to it, and a ring (3). When there is a relative movement between said first planet (21) and said ring (3) for a constant transmission ratio, a force of power transmission, Formula (I), between said first planet (21) and said ring (3) defines a plane (55). The transmission (10) comprises rolling means (15) for allowing a movement of translation between said ring (3) and said first planet (21) along a direction of translation (65) that is perpendicular to said plane (55) such that different transmission ratios can be obtained, corresponding to different coupling points (8) between said first lateral surface (31) and said ring (3) along said direction of translation (65).

### TECHNICAL FIELD

The invention relates to a continuously variable transmission or to a continuously variable drive train. More specifically, the invention relates to a planetary or to an epicyclic continuously variable transmission.

### DESCRIPTION OF RELATED ART

US2013/0296096A1 discloses a continuously variable transmission. In this drive train, there are two input disks coupled to an input shaft and separated by an output disk. Rollers are positioned between each input disk and the output disk for allowing a coupling between input disks and the output disk. Variation of the transmission ratio is achieved by varying the orientation of these rollers with respect to the input shaft and with respect to the input and output disks. A complex mechanism (comprising hydraulic reaction pistons for instance) is needed for varying the orientation of the rollers in an efficient and robust way. Such a mechanism is expensive. For allowing an efficient torque transmission between input disks and the output disk, a high pressure needs to be applied in order to have a required firm and close contact between the rollers, the input disks and the output disk. Such a high pressure is applied for instance on the external surface of one of the input disk by a hydraulic cylinder positioned at one hand of the drive train. Such a hydraulic cylinder requires some fluid. This increases the complexity of the train and its maintenance. It also increases the cost of the drive train. Due to this high pressure, a stationary variation of the transmission ratio is not possible. A stationary variation of the transmission ratio means a variation of transmission ratio when input shaft (and therefore input disks) and output disk do not rotate. For varying the orientation of the rollers, input and output disks must rotate. Another disadvantage of this drive train is that it is difficult to miniaturize it because of the complex system for moving the orientation of the rollers and because of these rollers. Last, this drive train is heavy.

WO2007/061993 A2 discloses a planetary continuously variable transmission that is known by the one skilled in the art as ‘NuVinci® Technology’. This transmission comprises two rings, generally acting as input and output of the transmission. Spheres having the role of the planets of a planetary transmission are coupled to these two rings and are able to rotate around an axis of revolution. The orientation of this axis of revolution can be modified. For instance, one or more idler (sun of a planetary transmission) coupled to these spheres can translate, imposing a modification of the orientation of the axis of revolution of the spheres. It results different transmission ratios between input and output rings depending on the orientation of the axis of revolution of the spheres. For varying the transmission ratio, ie for varying the orientation of the spheres, they must rotate in order to permit overcoming the friction forces coupling the spheres and the rings without damaging the transmission. Therefore, a change of transmission ratio when the input of the transmission is at rest is not possible. In other words, the NuVinci® transmission is not a stationary planetary continuously variable transmission.

For some applications, a stationary variation of transmission ratio is desired. This is notably the case for variable transmissions used in prostheses, for instance in an ankle prosthesis. Indeed, as explained in the publication “Variable Stiffness Actuator Based on Infinitely Variable Transmission: Application to an Active Ankle Prosthesis (Everarts, C. et al.)”, the energy efficiency of such a prosthesis is increased when a Continuously Variable Transmission is used. This transmission must be compact and able to vary its ratio during the whole gait cycle even during the phases of the gait cycle where the ankle velocity is zero, i.e. the prosthesis is not moving.

### SUMMARY OF THE INVENTION

It is an object of the invention to provide a stationary continuously variable transmission. To this end, the inventors propose a continuously variable planetary transmission comprising:
a sun;
a planet carrier;
a first planet mechanically coupled to said planet carrier and to said sun, presenting a first axis of revolution, and having a first lateral surface that is nonparallel to said first axis of revolution;
a ring coupled to said sun and said planet carrier through said first planet; said planet carrier and said first planet being configured such that a relative movement of rotation between them around said first axis of revolution is possible;
   said continuously variable planetary transmission being configured such that a coupling point between said ring and said first lateral surface is able to follow a plane curve in a plane when there is a relative movement between said first planet and said ring for a constant transmission ratio of said continuously variable planetary transmission.
   The continuously variable planetary transmission of the invention is characterized in that it comprises rolling means for allowing a movement of translation between said ring and said first planet along a direction of translation that is perpendicular to said plane such that said continuously variable planetary transmission presents different transmission ratios, corresponding to different coupling points between said first lateral surface of said first planet and said ring along said direction of translation.

Rolling means are therefore rigid in a direction along which force is transmitted between the first planet and the ring and free in a perpendicular direction (ie free along said direction of translation). In other words, rolling means are rigid in the direction of transmission of the effort between the first planet and the ring and free to roll in a direction perpendicular to the direction of transmission of said effort. It is therefore possible to vary at rest along this direction of translation the position of the coupling point between the first planet (or its first lateral surface) and the ring for varying the transmission ratio between them. The transmission of the invention is therefore a stationary continuously variable transmission. Thanks to the use of the rolling means, friction is high in one direction (direction of transmission of the effort between the first planet and the ring) and low in a perpendicular direction (ie along the direction of translation).

First lateral surface is nonparallel to the first axis of revolution. Such a feature is known by the one skilled in the art. Hence, the minimal distance between said first lateral surface and said first axis of revolution is not constant along said first axis of revolution. One could also say that first axis of revolution is not perpendicular to all the normal vectors of said first lateral surface. Or equivalently, that first axis of revolution is not parallel to all tangential planes of said first lateral surface.

With respect to US2013/0296096A1, the transmission of the invention has the following advantages. Change of transmission at rest is possible; this is not possible for the transmission of US2013/0296096A1. Also, change of transmission ratio at low speed is not possible with the transmission of US2013/0296096A1. Indeed, this transmission uses a fluid named ‘traction fluid’ by the one skilled in the art. A movement sufficient rapid of the different elements is necessary for this traction fluid' to work properly. This is not the case with the transmission of the invention that generally does not need such a ‘traction fluid’. Hence, the transmission of the invention allows changing the transmission ratios at low speed. Change of transmission ratios is simpler with the invention that only requires a movement of translation between the first planet and the ring. With the transmission of US2013/0296096A1, a synchronization of the orientations of the different rollers (six rollers in general) is necessary. The transmission of US2013/0296096A1 also requires imposing a large pressure between input disks, rollers, and output disk. This increases the cost. The transmission of the invention is more compact than the transmission of US2013/0296096A1. In this last transmission, the system allowing modifying the orientation of the rollers is not located along main axis of the transmission. Moreover, input and output shafts are not aligned, also increasing the required size for the transmission. The transmission of the invention does not require a hydraulic system; it is a purely mechanical device. Hence, the invention is simpler.

The transmission of the invention presents higher energy efficiency than other transmissions, in particular a higher energy efficiency than the transmissions disclosed in W02007/061993 A2 and US2013/0296096A1. For varying a transmission ratio with the transmission of the invention, one does not need to overcome a friction force, such as the friction force that exists between the spheres and the rings of NuVinci® technology. Indeed, the rolling means of the transmission of the invention allow having rolling without slipping when the transmission ratio is varied. In addition, the rotating elements of the transmission of the invention can present a lower inertia than the rotating elements of the transmissions of WO2007/061993 A2 and US2013/0296096A1. This allows varying the speeds of rotation rapidly with less energy. Rolling means of small cost can be used with the transmission of the invention. This leads to a transmission that is cheaper than NuVinci® technology. The transmission of the invention is also simpler than NuVinci® technology further decreasing its cost of fabrication. The mechanism used in the transmission of the invention is less complex than the one used in NuVinci® technology. The transmission of the invention does not need a fluid for transmitting torque contrary to NuVinci® technology for instance. The transmission of the invention does not need synchronization between different elements contrary to NuVinci® technology for instance. For these different reasons, the transmission of the invention is simpler.

With respect to a classical planetary transmission, the one of the invention has the advantage of allowing a continuous variation of the transmission ratio.

Preferably, the rolling means are coupled to said ring.

Preferably, the first lateral surface of the first planet is a first external lateral surface of said first planet.

Preferably, the transmission of the invention comprises at least one ball screw for controlling and precisely knowing the relative position between the first planet and the ring. More preferably, the transmission of the invention comprises three synchronized ball screws. Then, blocking risks are minimized. Rather than a ball screw, the transmission of the invention could comprise, according a preferred embodiment, any linear mechanism such as for instance: hydraulic or pneumatic actuator, linear motor, thread screw.

Preferably, said first planet has a smooth first lateral surface. With this preferred embodiment, a smooth variation of transmission ratio is easier. Moreover, with this preferred embodiment, a continuous range of transmission ratio is obtained more easily. If there were teeth on the first lateral surface, the pitch should stay constant for allowing an efficient coupling with the ring. But it is possible to put an integer number of teeth only for a discrete number of diameters perpendicular to said first axis of revolution. By using a smooth first lateral surface, all the possible values of such diameters of said first lateral surface can be used for transmitting power between first planet and the ring.

Preferably, said rolling means comprise rollers, each of them being able to roll around a roll axis that is perpendicular to said direction of translation. Rollers are simple elements. This preferred embodiment allows having a transmission that is easy to fabricate and that is cheap.

Preferably, said rollers have a shape of a diabolo. By using such rollers, one can obtain a substantially circular path for the plane curve along which said coupling point between said ring and said first lateral surface moves when there is a relative movement between first planet and ring for a constant transmission ratio. It follows a still higher energetic efficiency.

Preferably, said first axis of revolution presents an inclination of 45° with respect to said direction of translation. This preferred embodiment allows obtaining a particularly compact transmission that can present moreover large variations of the transmission ratio.

Preferably, said first planet has a shape of a right circular cone.

With this preferred embodiment, the fabrication of the planets and hence of the whole continuously variable planetary transmission is easier. The costs of fabrication can therefore be further reduced.

Preferably, said first planet has a shape of truncated right circular cone. This preferred embodiment allows having the advantages of using a first planet that has a shape of a right circular cone, but that is more compact.

Preferably, said first planet having the shape of a right circular cone or a truncated right circular cone has an aperture angle of 90°. This preferred embodiment allows having the advantages of using a first planet that has a shape of a right circular cone, and that is particularly compact.

One can use various types of mechanical couplings between said first planet and said sun. According to a first example, friction transmission between these two elements can be used. Alternatively, one can use a bevel gear mechanism for coupling said first planet and said sun. These two alternatives allow reducing the size of the transmission.

Preferably, said first planet is mounted around a shaft parallel to said first axis of revolution with a translational degree of freedom along said shaft. This preferred feature allows having a more efficient coupling between first planet and ring even when said plane curve is not strictly circular.

Preferably, the continuously variable planetary transmission comprises pushing means for pushing said first planet towards said ring. This preferred embodiment allows improving coupling between first planet and ring. Preferably, these pushing means comprise springs.

Preferably, the continuously variable planetary transmission further comprises a second planet mechanically coupled (e.g. bevel gear, friction coupling) to said planet carrier and to said sun, presenting a second axis of revolution, having a second lateral surface that is nonparallel to said second axis of revolution; said ring being also coupled to said sun and said planet carrier through said second planet; said planet carrier and said second planet being configured such that a relative movement of rotation between them around said second axis of revolution is possible; said continuously variable planetary transmission being configured such that a coupling point between said ring and said second lateral surface is able to follow a plane curve in a plane when there is a relative movement between said second planet and said ring for a constant transmission ratio of said continuously variable planetary transmission; said continuously variable planetary transmission comprising rolling means for allowing a movement of translation between said ring and said second planet along a direction of translation that is perpendicular to said plane such that said continuously variable planetary transmission presents different transmission ratios, corresponding to different coupling points between said second lateral surface of said second planet and said ring along said direction of translation.

By using two or more planets, one can reduce their size. The torque each planet has to support is then also reduced. Preferably, the second lateral surface is smooth.

Preferably, said second lateral surface is a second external lateral surface of said second planet.

Preferably, the continuously variable planetary transmission comprises four planets, ie a first, a second, a third, and a fourth planets.

Preferably, the continuously variable planetary transmission comprises four planets. Preferably, said rolling means comprise six rollers. The inventors found that using four planets and six rollers is particularly well-adapted for practical cases. Moreover, such a preferred embodiment has the further following advantage. When two planets are in contact with the boundaries between two rollers, two other planets can be brought in contact with a central portion of two rollers (and this, for all possible angular position of the planets with respect to the ring and to the rollers). This provides no discontinuity in the torque transmission between the planets and the ring because of the displacement of the contact points between the planets and the different rollers. Finally, the efficiency of the transmission is increased.

Preferably, said ring presents an axis of symmetry that is parallel to said direction of translation. Then, the transmission of the invention can be still more compact.

Preferably, there is a friction fluid (or transmission fluid) between said first planet (between the different planets when more than one planet is used) and said ring for coupling the first lateral surface of said first planet to said ring.

Preferably, the continuously variable planetary transmission further comprises a second planetary stage comprising: a sun; a second planetary stage ring mechanically coupled to said planet carrier; a second planetary stage planet carrier; a second planetary stage first planet coupled to said second planetary stage planet carrier, to said second planetary stage ring, and to said sun.

This preferred embodiment allows having an infinitely variable transmission or IVT. This term is known by the one skilled in the art. An IVT is a mechanism which allows varying a transmission ratio between an output and an input from negative to positive values continuously, passing through a zero value.

Preferably, the sun of said second planetary stage is identical to the sun of the continuously variable planetary transmission of first planetary stage that has been described first.

By using a same sun for the second stage, one can reduce the costs of fabrication of the transmission.

Preferably, the sun of said second planetary stage is different from the sun of the continuously variable planetary transmission of first planetary stage that has been described first, and said two suns are mechanically coupled.

Then, one could impose a speed of rotation that is different for these two suns, for example by using gears for coupling these two suns. This allows increasing the range of transmission ratios that the transmission of the invention can propose.

Preferably, the invention relates to a prosthesis comprising a continuously variable planetary transmission as described before, including all its possible preferred embodiments. This prosthesis has an improved energetic efficiency with respect to prosthesis's known by the one skilled in the art.

### BRIEF DESCRIPTION OF THE DRAWING

These and further aspects of the invention will be explained in greater detail by way of example and with reference to the accompanying drawings in which:

FIG. 1 schematically shows a preferred embodiment of the transmission of the invention;

FIG. 2 shows in a simplified manner coupling between first planet and ring;

FIG. 3 schematically shows a planetary transmission;

FIG. 4 shows a two-dimensional cross-section of an example of a ring and of rollers coupled to it;

FIG. 5 shows a perspective view of a preferred planet carrier and of a sun;

FIG. 6 shows a perspective view of an example of first, second, third and fourth planets coupled to a planet carrier;

FIG. 7 shows a perspective view of the transmission when the rolling means comprise rollers having a shape of a diabolo;

FIG. 8 shows a perspective view of an example of the transmission;

FIG. 9 schematically shows the transmission of the invention according to another preferred embodiment;

FIG. 10 shows a preferred mechanism for moving the ring with respect to the planet(s) and for controlling its position.

The drawings of the figures are neither drawn to scale nor proportioned. Generally, identical components are denoted by the same reference numerals in the figures.

### DETAILED DESCRIPTION OF PREFERRED EMBODIMENTS OF THE INVENTION

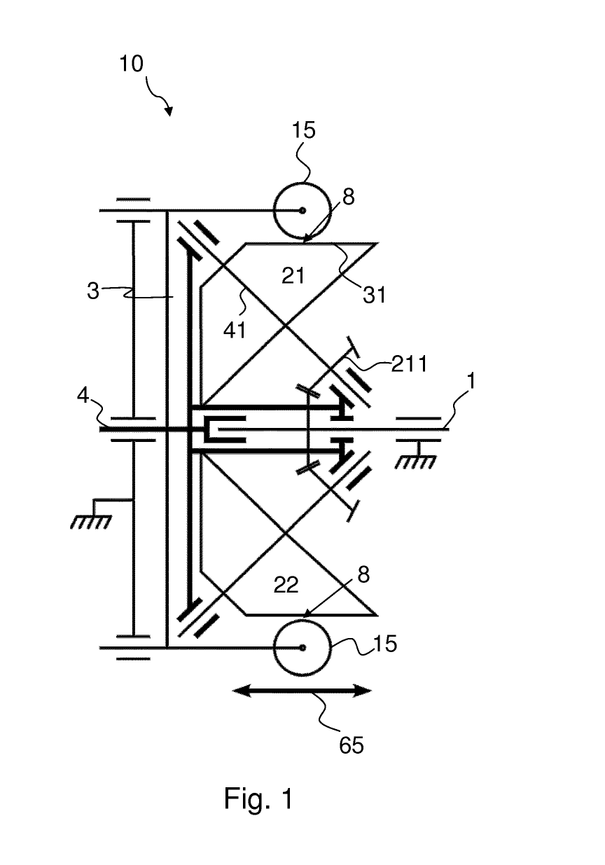

FIG. 1 schematically shows the continuously variable planetary transmission 10 (or transmission 10) of the invention according to a preferred embodiment. It comprises a sun 1, or a sun gear member, or a sun gear part, or a sun gear mechanism. Preferably, the sun 1 comprises a sun shaft and a sun gear. Then, the sun shaft can be the output shaft or the input shaft of the transmission 10 in some preferred examples. The transmission 10 also comprises a planet carrier 4, or a planet carrier member, or a planet carrier part, or a planet carrier mechanism. Preferably, this planet carrier 4 is connected to a drive shaft or a motor. In such a case, planet carrier 4 is therefore input of the transmission 10. In another preferred embodiment, planet carrier 4 can nevertheless be connected to the output.

The transmission 10 further includes a first planet 21. This first planet 21 presents a first axis of revolution 41. In other words, first planet 21 is a solid of revolution. These terms are known by the one skilled in the art. They mean that first planet 21 is a solid that can be obtained by rotating a plane curve around said first axis of revolution 41 that lies in the same plane as said plane curve. First lateral surface 31 represents the surface that is generated by said plane curve when rotating it around said first axis of revolution 41. The meaning or main ideal of the invention is not modified if first axis of revolution 41 is not strictly an axis of revolution because of small variations of the first lateral surface 31 around said first axis of revolution 41. Therefore, one could say that first axis of revolution 41 is substantially an axis of revolution of first planet 21. Preferably, first lateral surface 31 is smooth. First planet 21 is mechanically coupled to the planet carrier 4 and to the sun 1. As shown in FIG. 1, coupling between sun 1 and first planet 21 is preferably realized through a bevel gear mechanism or through a bevel planet-sun gear 211. This allows having a sun 1 or sun shaft that is not parallel to first axis of revolution 41 of first planet 21. As shown in the preferred embodiment of FIG. 1, first planet 21 preferably has a shape of a truncated cone. Other shapes are nevertheless possible. A relative movement of rotation between planet carrier 4 and first planet 21 is possible around first axis of revolution 41. Preferably, the transmission 10 has more than one planet. In the preferred example of FIG. 1, transmission 10 has two planets: a first 21 and a second 22 planets.

The transmission 10 also comprises a ring 3. Said ring 3 is coupled to the sun 1 and to the planet carrier 4 through first planet 21 (and through a second planet 22 in the example of FIG. 1). Indeed, as shown in FIG. 1, there is a coupling point 8 between the ring 3 and first lateral surface 31 of first planet 21. As the transmission 10 of FIG. 1 also comprises a second planet 22, there is also a coupling point 8 between the ring 3 and the second lateral surface of second planet 22.

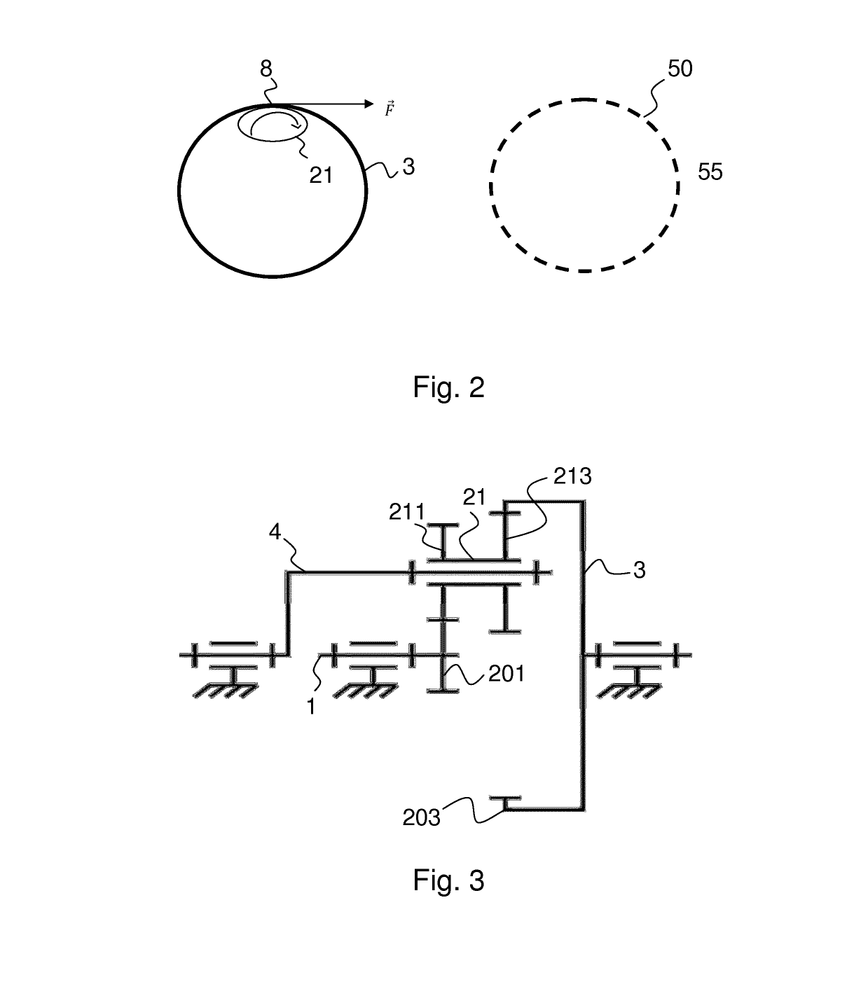

FIG. 2 shows in a simplified manner coupling between first planet 21 and ring 3, in a 2D simplified view. When the transmission 10 of the invention presents a constant (or fixed, determined) transmission ratio, and when there is a relative movement between first planet 21 and ring 3, there is a force \vec{F} transmitting power (that we name force of power transmission, \vec{F}, in the following) between said ring 3 and said first planet 21. This force of power transmission, \vec{F}, is applied at the coupling point 8 between said ring 3 and said first planet 21 and is tangential to first lateral surface 31 of first planet 21. If the ring 3 is fixed in rotation, this force \vec{F} induces first planet 21 to rotate around its first axis of revolution 41. If force of power transmission, \vec{F}, is applied by the ring 3 on first planet 21 (for instance by reaction), the latter then rotates in the direction illustrated by the arrow lying inside first planet 21. Hence, a torque is transmitted between first planet 21 and ring 3. Because of the rotation of the first planet 21, the coupling point 8 between said ring 3 and said first planet 21 follows a path that is a plane curve 50. For the example of FIG. 2, this plane curve 50 is a circle (see right part of same FIG. 2). This plane curve 50 lies in a plane 55 that is the plane of right part of FIG. 2. As the coupling point 8 follows the plane curve 50, the direction of the force of power transmission, \vec{F}, changes. The different directions of the force of power transmission, \vec{F}, are also included in the plane 55 of right part of FIG. 2. The force of power transmission, \vec{F}, could also be named tangential force \vec{F} between ring 3 and first planet 21.

In the preferred embodiment shown in FIG. 1, the coupling point 8 allows a mechanical coupling between first planet 21 and a roller 15 that is mechanically coupled to the ring 3 (and hence, finally, a mechanical coupling between first planet 21 and ring 3 is possible through coupling point 8). In the example of FIG. 1, the transmission 10 indeed comprises rolling means 15 such as rollers that are preferably coupled to the ring 3. These rolling means 15 allow having a movement of translation between the ring 3 and the first planet 21 along a direction of translation 65 that is perpendicular to the plane 55 shown in right part of FIG. 2. Hence, the direction of translation 65 is perpendicular to the plane 55 comprising the plane curve 50. This direction of translation 65 is also perpendicular to the force of power transmission, \vec{F} (or to the tangential force \vec{F} between ring 3 and first planet 21), shown in FIG. 2 for each position of the coupling point 8 along the plane curve 50 (provided that the transmission 10 has a constant transmission ratio). In other words, the direction of translation 65 is perpendicular to the tangential force \vec{F} that can develop between ring 3 and first planet 21 for a fixed transmission ratio of the transmission 10. When there is such a movement of translation between the ring 3 and first planet 21, the coupling point 8 between said ring 3 and said first lateral surface 31 of first planet 21 varies along said direction of translation 65. As a consequence, transmission ratio between first planet 21 and planet carrier 4 then varies. In other words, by varying said coupling point 8 along the direction of translation 65, one obtain different values for the ratio of speeds of rotation between first planet 21 and planet carrier 4. And this variation is continuous. This allows having a continuously variable transmission 10 as it is explained below.

FIG. 3 schematically shows a planetary transmission comprising a sun 1, a planet carrier 4, a first planet 21, and a ring 3. First planet 21 is coupled to the sun 1 through a planet-sun gear 211, and through a sun gear 201. Planet-sun gear 211 is for instance a bevel planet-sun gear 211 as shown in FIG. 1. First planet 21 is coupled to the ring 3 through a planet-ring gear 213, and through a ring gear 203. Planetary gear kinematic equation is:

$$\frac{{ω}_{1} - {ω}_{4}}{{ω}_{3} - {ω}_{4}} = - \frac{{Z}_{2} {Z}_{3}}{{Z}_{2}^{′} {Z}_{1}} . \qquad ( Eq . 1 )$$

In equation (Eq. 1), ωᵢ stands for the angular velocity of element i, where element i can be the sun 1, the ring 3, and the planet carrier 4. Z₂ rimitive diameter of planet-sun gear 211; Z′₂ primitive diameter of planet-ring gear 213; Z₃ rimitive diameter of ring gear 203; Z₁ rimitive diameter of sun gear 201. The terms ‘primitive diameter’ are known by the one skilled in the art. By defining:

R_{B}=^{Z}^{₂}/_{Z}_{₁};

R_{C}=^{Z}^{₃}/_{Z′}_{₂};

equation (Eq. 1) becomes:

$$\frac{{ω}_{1} - {ω}_{4}}{{ω}_{3} - {ω}_{4}} = - {R}_{B} {R}_{C} . \qquad ( Eq . 2 )$$

Hence, by continuously varying R_{C}, ie by continuously varying Z′₂ for instance, one can modify the ratio

$$\frac{{ω}_{1} - {ω}_{4}}{{ω}_{3} - {ω}_{4}} .$$

By using the transmission 10 of the invention, Z′₂ can be continuously varied by imposing a movement of translation between the ring 3 and the first planet 21 along the direction of translation 65.

For illustrative purposes, let us assume that the ring 3 is fixed, then ω₃ =0. If the sun 1 is the output, and if the planet carrier 4 is the input, equation (Eq. 2) becomes:

$$\frac{{ω}_{0} - {ω}_{i}}{0 - {ω}_{i}} = - {R}_{B } {R}_{C} . \qquad ( Eq . 3 )$$

### Hence,

ω₀=ωᵢ(1+R_{B}R_{C}) (Eq. 4),

where ω₀=ωₗ represents the speed of the output or output shaft, and where ωᵢ=ω₄ represents the speed of the input or input shaft. By continuously varying R_{C}, ie by continuously varying Z′₂ for instance, one can continuously modify ω₀.

Elements other than the ring 3 could be fixed. For instance, sun 1 or planet carrier 4 could be fixed. Also, input and output could be other elements than the planet carrier 4 and the sun 1. For instance, ring 3 could be input or output.

The terms continuously variable are known by the one skilled in the art. They mean that there are no fixed gear ratio. In other words, the transmission 10 of the invention can change seamlessly through an infinite number of transmission ratios as shown by equation (Eq. 4) for instance. Transmission ratio is defined as the ratio between angular speeds of output and input. By using the convention of equation (Eq. 4), transmission ratio is therefore equal to ^{ω}^{₀}/_{ω}_{ᵢ}i. With the transmission 10 of the invention, transmission ratio comprised for instance between 1 and 15 can be obtained. Other values of transmission ratio such as values between 3 and 11 can be obtained.

Preferably, there is a planet carrier shaft mechanically coupled to the planet carrier with a transmission ratio of 1/1.

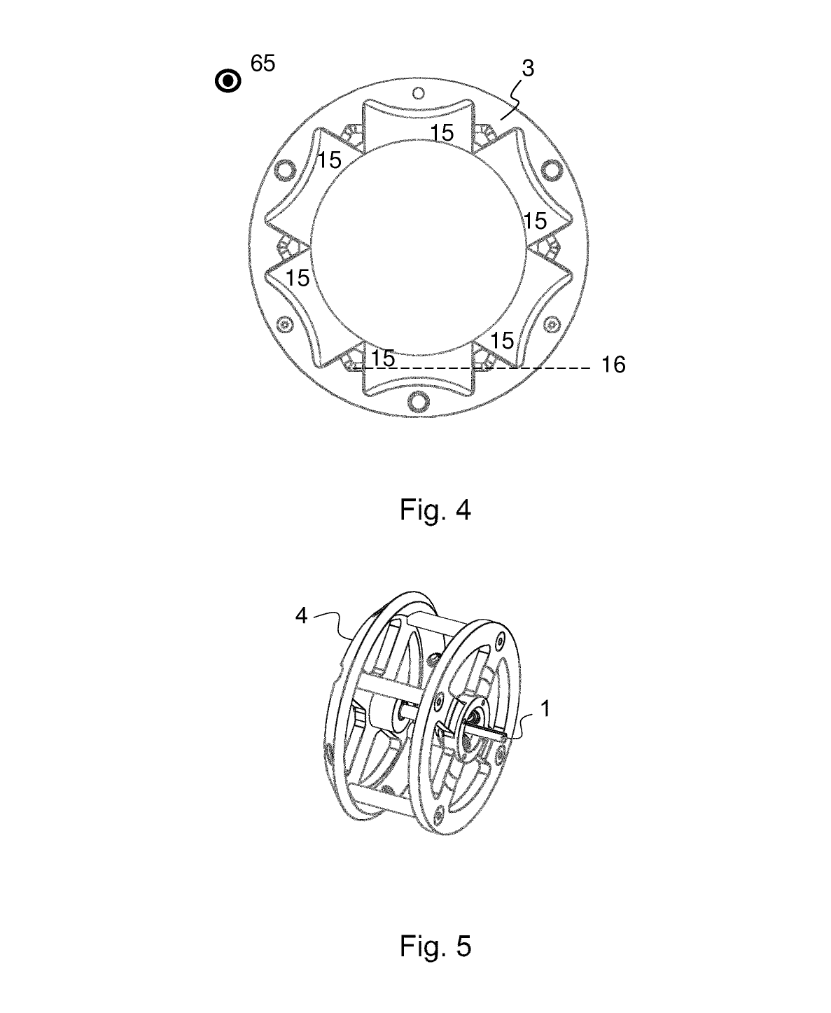

Preferably, rolling means 15 comprise rollers. FIG. 4 shows a 2D cross-section of the ring 3 and of rollers 15 coupled to said ring 3. As shown in this figure, there are preferably six rollers 15. Each roller 15 is able to roll around a roll axis 16 that is perpendicular to the direction of translation 65 defined above. Hence, these roll axes 16 around which the rollers 15 can rotate are located in the plane 55 defined above in relation to right part of FIG. 2 or in a parallel plane. As shown in FIG. 4, rollers preferably have a shape of diabolo. A diabolo has symmetry of revolution and presents a central diameter of revolution that is smaller than the diameter of revolution at its extremities along the axis of revolution. By using rollers having a shape of a diabolo, one can have a plane curve 50 that tends to be a circle. For some shapes of diabolo, it is even possible to have a substantially circular plane curve 50. As a reminder, this plane curve 50 is the curve along which the coupling point 8 between the ring 3 and the first planet 21 moves when transmission ratio of the transmission 10 is constant, and when there is a relative movement between first planet 21 and the ring 3. Thanks to the fact that said plane curve 50 tends to be a circle, energetic efficiency of the transmission 10 is improved.

Preferably, rolling means 15 comprise rollers able to roll around a roll axis 16 that is perpendicular to the direction of translation 65 defined above, each of them having a shape of a hyperboloid of revolution. A diabolo can have a shape of a hyperboloid of revolution, but a diabolo does not have necessarily a shape of hyperboloid of revolution. By using rollers having a shape of a hyperboloid of revolution, energetic efficiency of the transmission 10 is improved.

Preferably, first axis of revolution 41 of first planet 21 presents an inclination of 45° with respect to said direction of translation 65. The preferred embodiment shown in FIG. 1 includes this preferred feature. When planet carrier 4 rotates around its axis of rotation (which is parallel to the direction of translation 65 in the preferred embodiment of FIG. 1), first axis of revolution 41 undergoes a movement of precession around this axis of rotation of planet carrier 4. But for each of its positions, first axis of revolution 41 defines an angle of 45° with the direction of translation 65.

As shown in FIG. 1, first planet 21 preferably has a shape of truncated right circular cone with an aperture angle of 90°. This angle is the maximum angle between two generatrix lines of the cone from which the truncated cone is extracted. This angle is also equal to two times the angle a generatrix line makes with the first axis of revolution 41 of the cone.

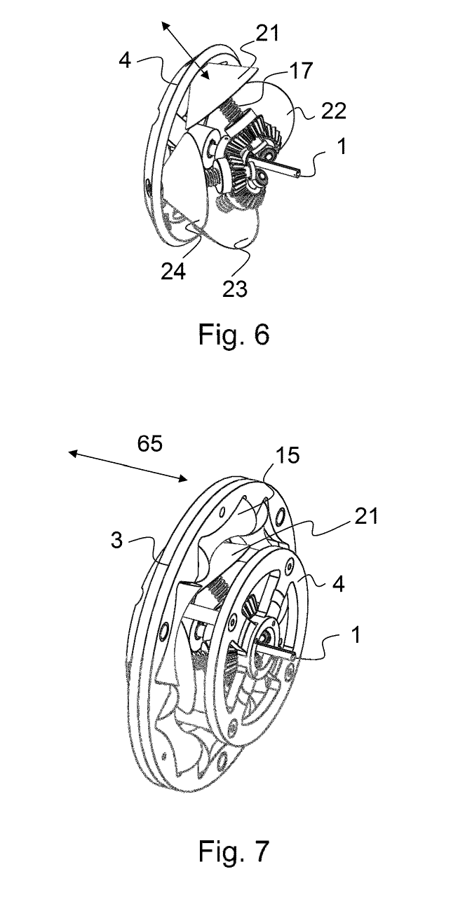

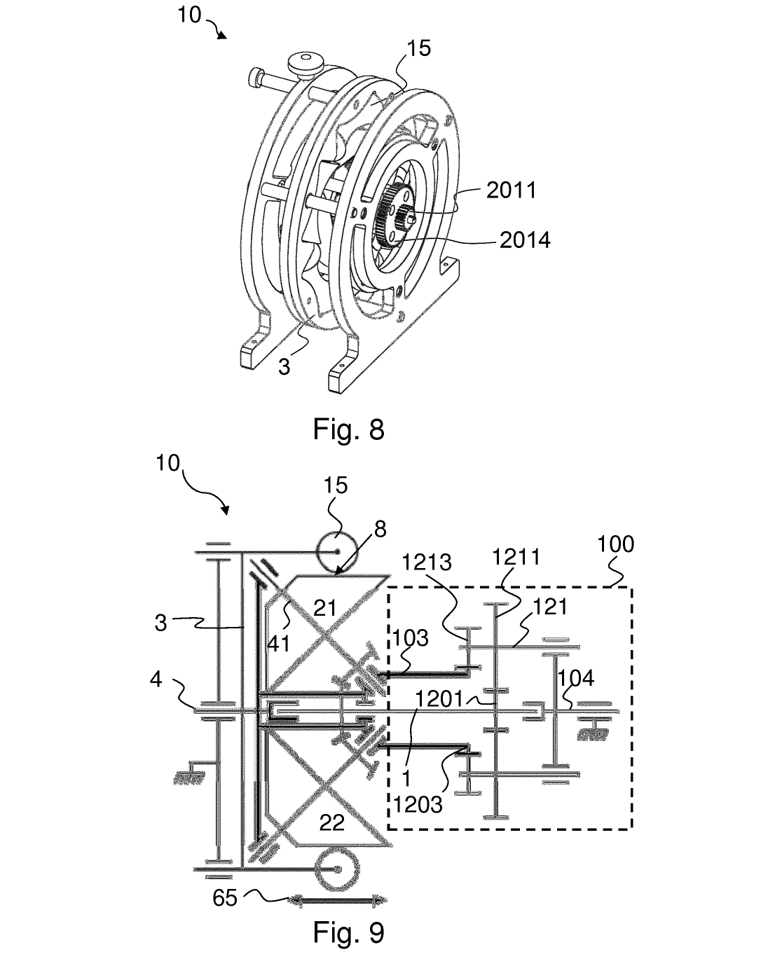

FIGS. 5 to 8 show perspective views of an example of the transmission 10 of the invention. FIG. 5 shows the planet carrier 4 and the sun 1. The planet carrier 4 has a shape of a cage. This planet carrier 4 presents symmetry with respect to the sun 1 that has a shape of a shaft. As shown in FIG. 6, the example of the transmission 10 comprises a first 21, a second 22, a third 23, and a fourth 24 planets. For clarity reasons, only the left part of the planet carrier 4 is shown in this figure. The planets (21, 22, 23, 24) are mounted on the planet carrier 4 and geared with the sun 1 through bevel planet-sun gears. As shown in FIG. 6, each planet (21, 22, 23, 24) has preferably a shape of a truncated right circular cone in this example. We can also see that each planet (21, 22, 23, 24) preferably has a smooth lateral surface (or smooth external surface which is intended to be coupled to the ring 3). Each planet (21, 22, 23, 24) is mounted on a shaft with a translational degree of freedom along said shaft. Hence, first planet 21 for instance, can move along the double arrow shown in FIG. 6. This translational degree of freedom is nevertheless constrained with pushing means 17, for instance a spring, in order to add compliance and maintain a known force at the coupling point 8 between planets (21, 22, 23, 24) and ring 3. The ring 3 can be visualized in FIG. 7. Rollins means 15 coupled to said ring 3 allow it to move along the direction of translation 65. In the example shown in FIG. 7, rolling means comprise six diabolo-like rollers. Hence, there is low friction in a direction parallel to the sun shaft and high friction is a perpendicular or tangential direction. The torque that the transmission 10 can transmit is notably fixed by the compression force of the pushing means 17, and by the friction coefficient between planets (21, 22, 23, 24) and rollers. FIG. 8 shows a preferred final assembly for this example of transmission 10. As illustrated in this figure, the transmission 10 preferably has an output sun gear 2011 mechanically coupled with the sun 1, and an output planet carrier gear 2014 mechanically coupled with the planet carrier 4. Preferably, output sun gear 2011 (respectively output planet carrier gear 2014) is directly connected to said sun 1 (respectively to said planet carrier 4) such that they have same angular velocities. The roles of input and output can be varied. As it can be seen in FIGS. 5 to 8, the transmission 10 preferably presents symmetry with respect to the sun 1 or sun shaft.

Preferably, the transmission 10 of the invention comprises a second planetary stage 100. An example of this preferred embodiment is schematically shown in FIG. 9. The second planetary stage 100 comprises a sun 1. In the example shown in FIG. 9, the sun of the second planetary stage 100 is same sun 1 of the one described above. One could nevertheless use another sun for the second planetary stage 100 and couple it to the sun 1 described above. Coupling between such two suns 1 can be such that they present same or different angular velocities. The second planetary stage 100 also comprises a second planetary stage ring 103 mechanically coupled to the planet carrier 4 described above. Coupling between second planetary stage ring 103 and the planet carrier 4 described above can be such that they present same or different angular velocities. The second planetary stage 100 also comprises a second planetary stage planet carrier 104, and a second planetary stage first planet 121. This last second planetary stage first planet 121 is coupled to said second planetary stage planet carrier 104, to said second planetary stage ring 103 and to said sun 1. By using such a preferred embodiment of the transmission 10, comprising this second planetary stage 100, one can obtain an IVT as it is explained below.

For the second planetary stage 100, equation (Eq. 1) becomes:

$$\frac{{ω}_{0} - {ω}_{out}}{{ω}_{i} - {ω}_{out}} = {R}_{S} {R}_{CR} \qquad ( Eq . 5 )$$

where:
ω₀=ω₁, ie angular velocity of sun 1;
ωₒᵤₜ=ω₁₀₄, ie angular velocity of second planetary stage planet carrier 104;
ωᵢ=ω₁₀₃, ie angular velocity of second planetary stage ring 103; in the preferred example shown in FIG. 9, ie angular velocity of second planetary stage ring 103 is equal to angular velocity of planet carrier 4;

R_{S}=^{ZZ}^{₂}/_{ZZ}_{₁};

R_{CR}=^{ZZ}^{₃}/_{ZZ′}_{₂}.

ZZ₂ is primitive diameter of planet-sun gear 1211 of second planetary stage first planet 121; ZZ, is primitive diameter of sun gear 1201 at second stage 100; ZZ₃ is primitive diameter of second planetary stage ring gear 1203; ZZ′₂ is primitive diameter of planet-ring gear 1213 of second planetary stage first planet 121. There is no minus sign in right-hand part of equation (Eq. 5) because second planetary stage ring 103 is inside second planetary stage first planet 121; this inverts the sense of rotation of second planetary stage ring 103.

Equation (Eq. 5) can be rewritten:

$$\begin{matrix}{ω}_{0} - {ω}_{out} = {R}_{S} {R}_{CR} ( {ω}_{i} - {ω}_{out} ) , & ( Eq . 6 ) \\ ⇔ {ω}_{out} = \frac{{ω}_{0} - {ω}_{i} {R}_{S} {R}_{CR}}{1 - {R}_{S} {R}_{CR}} . & ( Eq . 7 )\end{matrix}$$

By using the expression of ω₀ given by equation (Eq. 4), one can obtain:

$$\begin{matrix}{ω}_{out} = \frac{{ω}_{i} ( 1 + {R}_{C} {R}_{B} ) - {ω}_{i} {R}_{S} {R}_{CR}}{1 - {R}_{S} {R}_{CR}} , & ( Eq . 8 ) \\ ⇔ {ω}_{out} = {ω}_{i} \frac{1 + {R}_{C} {R}_{B} - {R}_{S} {R}_{CR}}{1 - {R}_{S} {R}_{CR}} . & ( Eq . 9 )\end{matrix}$$

Hence, transmission 10 with second planetary stage 100 allows having a large spectrum of values for the transmission ratio, ¹⁰⁷ ^{ₒᵤₜ}/_{ω}_{ᵢ}, potentially comprising zero. In particular, negative and positive values of the transmission ratio can be obtained. This large spectrum of values is obtained by adjusting the ratios R_{B}, R_{S}, R_{CR}, R_{C}. In particular, by varying R_{C}, the numerator and denominator can take either same or opposite signs. Moreover, by varying R_{C}, the transmission ratio, ^{ω}^{ₒᵤₜ}/₁₀₇ _{ᵢ}, can change continuously across positive and negative values. Transmission 10 with second planetary stage 100 of FIG. 9 is therefore an IVT. As an example, one skilled in the art could choose the following values: R_{B}=1; R_{S}=R_{CR}=54/20; R_{C} varying between 4 and 20.

In equation (Eq. 9) we chose planet carrier 4 as input and second planetary stage planet carrier 104 as output. Other input and output could nevertheless be chosen such that the transmission 10 with the second planetary stage 100 is an IVT. For instance, when the transmission 10 is used with ankle prosthesis, an ankle is preferably connected to planet carrier 4 of first stage, and a motor is preferably connected to second planetary stage planet carrier 104 of second planetary stage 100.

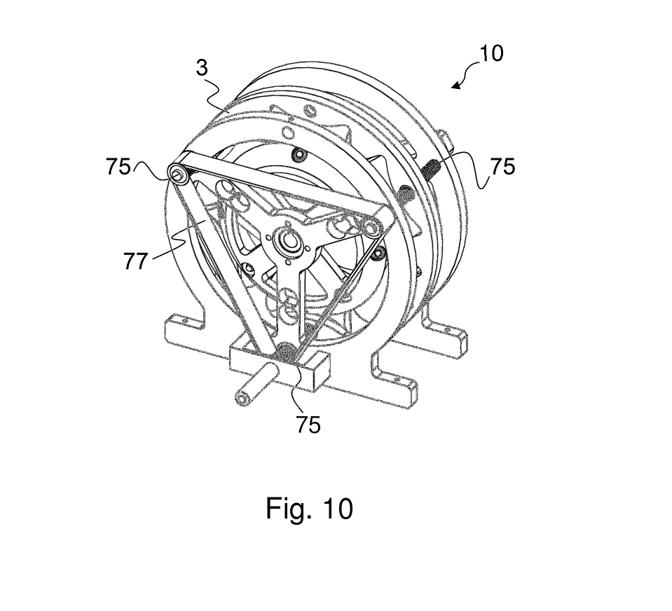

Different mechanisms can be used for moving the ring 3 with respect to the planet(s) (21; 22; 23; 24). One can use for instance one or more screws coupled to said ring 3 and such that it can move with respect to the screw(s). FIG. 10 shows a preferred mechanism comprising three ball screws 75. Then, the transmission 10 of the invention preferably comprises such three ball screws 75 coupled to the ring 3 and allowing controlling the position and the movement of translation of the ring 3 with respect to the planet(s) (21; 22; 23; 24). By using ball screws 75, a high precision positioning is possible. By using three ball screws 75, risks of jamming of the ring 3 are reduced. Preferably such ball screws 75 are synchronized, preferably with a belt 77 as shown in FIG. 10. Preferably, one of the ball screws 75 can be activated by a motor (not shown in FIG. 10) for varying the position of the ring 3 with respect to the planet(s) (21; 22; 23; 24). According to another preferred embodiment, the transmission comprise three motors that are synchronized, for instance by a controller.

The present invention has been described in terms of specific embodiments, which are illustrative of the invention and not to be construed as limiting. More generally, it will be appreciated by persons skilled in the art that the present invention is not limited by what has been particularly shown and/or described hereinabove. Reference numerals in the claims do not limit their protective scope. Use of the verbs “to comprise”, “to include”, or any other variant, as well as their respective conjugations, does not exclude the presence of elements other than those stated. Use of the article “a”, “an” or “the” preceding an element does not exclude the presence of a plurality of such elements. Different elements of the transmission 10 of the invention can be chosen fixed or mobile. Also, different elements of the transmission 10 can be output or input.

The invention can also be summarized as follows. Transmission 10 comprising a sun 1, a planet carrier 4, a first planet 21 having a first axis of revolution 41 and a first lateral surface 31 that is nonparallel to it, and a ring 3. When there is a relative movement between said first planet 21 and said ring 3 for a constant transmission ratio, a force of power transmission, \vec{F}, between said first planet 21 and said ring 3 defines a plane 55. The transmission 10 comprises rolling means 15 for allowing a movement of translation between said ring 3 and said first planet 21 along a direction of translation 65 that is perpendicular to said plane 55 such that different transmission ratios can be obtained, corresponding to different coupling points 8 between said first lateral surface 31 and said ring 3 along said direction of translation 65.

1. Continuously variable planetary transmission (10) comprising:
a sun (1);
a planet carrier (4);
a first planet (21):
mechanically coupled to said planet carrier (4) and to said sun (1),
presenting a first axis of revolution (41),
having a first lateral surface (31) that is nonparallel to said first axis of revolution (41);
a ring (3) coupled to said sun (1) and said planet carrier (4) through said first planet (21);
said planet carrier (4) and said first planet (21) being configured such that a relative movement of rotation between them around said first axis of revolution (41) is possible;
said continuously variable planetary transmission (10) being configured such that a coupling point (8) between said ring (3) and said first lateral surface (31) is able to follow a plane curve (50) in a plane (55) when there is a relative movement between said first planet (21) and said ring (3) for a constant transmission ratio of said continuously variable planetary transmission (10); characterized in that
said continuously variable planetary transmission (10) comprises rolling means (15) for allowing a movement of translation between said ring (3) and said first planet (21) along a direction of translation (65) that is perpendicular to said plane (55) such that said continuously variable planetary transmission (10) presents different transmission ratios, corresponding to different coupling points (8) between said first lateral surface (31) of said first planet (21) and said ring (3) along said direction of translation (65).

2. Continuously variable planetary transmission (10) according to claim 1 characterized in that said first planet (21) has a smooth first lateral surface (31).

3. Continuously variable planetary transmission (10) according to claim 1 characterized in that said rolling means (15) comprise rollers, each of them being able to roll around a roll axis (16) that is perpendicular to said direction of translation (65).

4. Continuously variable planetary transmission (10) according to claim 3 characterized in that said rollers have a shape of a diabolo.

5. Continuously variable planetary transmission (10) according to claim 1 characterized in that said first axis of revolution (41) presents an inclination of 45° with respect to said direction of translation (65).

6. Continuously variable planetary transmission (10) according to claim 1 characterized in that said first planet (21) has a shape of a right circular cone.

7. Continuously variable planetary transmission (10) according to claim 1 characterized in that said first planet (21) has a shape of truncated right circular cone.

8. Continuously variable planetary transmission (10) according to claim 6 characterized in that said right circular cone has an aperture angle of 90°.

9. Continuously variable planetary transmission (10) according to claim 1 characterized in that it comprises a bevel gear mechanism for coupling said first planet (21) and said sun (1).

10. Continuously variable planetary transmission (10) according to claim 1 characterized in that said first planet (21) is mounted around a shaft parallel to said first axis of revolution (41) with a translational degree of freedom along said shaft.

11. Continuously variable planetary transmission (10) according to claim 1 characterized in that it comprises pushing means (17) for pushing said first planet (21) towards said ring (3).

12. Continuously variable planetary transmission (10) according to claim 1 characterized in that:
it further comprises a second planet (22):
mechanically coupled to said planet carrier (4) and to said sun (1),
presenting a second axis of revolution (42),
having a second lateral surface (32) that is nonparallel to said second axis of revolution (42); in that
said ring (3) is also coupled to said sun (1) and said planet carrier (4) through said second planet (22); in that
said planet carrier (4) and said second planet (22) are configured such that a relative movement of rotation between them around said second axis of revolution (42) is possible; in that
said continuously variable planetary transmission (10) is configured such that a coupling point (8) between said ring (3) and said second lateral surface (32) is able to follow a plane curve (50) in a plane (55) when there is a relative movement between said second planet (22) and said ring (3) for a constant transmission ratio of said continuously variable planetary transmission (10); and in that
said continuously variable planetary transmission (10) comprises rolling means (15) for allowing a movement of translation between said ring (3) and said second planet (21) along a direction of translation (65) that is perpendicular to said plane (55) such that said continuously variable planetary transmission (10) presents different transmission ratios, corresponding to different coupling points (8) between said second lateral surface (32) of said second planet (22) and said ring (3) along said direction of translation (65).

13. Continuously variable planetary transmission (10) according to claim 1 characterized in that it comprises four planets (21; 22; 23; 24).

14. Continuously variable planetary transmission (10) according to claim 1 characterized in that said rolling means (15) comprise six rollers.

15. Continuously variable planetary transmission (10) according to claim 1 characterized in that said ring (3) presents an axis of symmetry (13) that is parallel to said direction of translation (65).

16. Continuously variable planetary transmission (10) according to claim 1 characterized in that it further comprises a second planetary stage (100) comprising:
a sun (1);
a second planetary stage ring (103) mechanically coupled to said planet carrier (4);
a second planetary stage planet carrier (104);
a second planetary stage first planet (121) coupled to said second planetary stage planet carrier (104), to said second planetary stage ring (103), and to said sun (1).

17. Continuously variable planetary transmission (10) according to claim 16 characterized in that said sun of said second planetary stage (100) is identical to the sun (1) of the continuously variable planetary transmission (10) of any of claims 1 to 15.

18. Continuously variable planetary transmission (10) according to claim 16 characterized in that said sun of said second planetary stage (100) is different from the sun (1) of the continuously variable planetary transmission (10) of any of claims 1 to 15, and in that said two suns are mechanically coupled.

19. Prosthesis comprising a continuously variable planetary transmission (10) according to claim 1.
